(19) United States
(12) Patent Application Publication
(10) Pub. No.: US 2026/0083475 A1
(43) Pub. Date: Mar. 26, 2026

(54) SAFE SUCTION TIP

(71) Applicant: Daniel R. Jacobson, Chicago, IL (US)

(72) Inventor: Daniel R. Jacobson, Chicago, IL (US)

(21) Appl. No.: 19/337,503

(22) Filed: Sep. 23, 2025

Related U.S. Application Data
(60) Provisional application No. 63/698,044, filed on Sep. 24, 2024.

(51) Int. Cl. A61B 17/34; A61F 13/38; A61F 13/44; A61M 1/00

(52) U.S. Cl. CPC A61B 17/3421; A61F 13/38; A61F 13/44; A61M 1/77; A61B 2017/3437; A61B 2017/3454

(57) ABSTRACT

Disclosed are devices, systems and methods for a removeable and replaceable tip for a surgical (or dental) suction cannula which facilitates the safe and effective removal of blood and other bodily fluids from a body cavity while allowing manipulation and/or management of various tissues therein. These devices protect delicate anatomic structures from excessive negative pressure while allowing effective suctioning of fluid and debris from the operative field.

### CROSS-REFERENCE TO RELATED APPLICATIONS

This application claims the benefit of U.S. Provisional Application No. 63/698,044 entitled “SURGICAL SUCTION DEVICES” filed Sep. 24, 2024, the disclosure of which is incorporated by reference herein in its entirety.

### FIELD OF THE INVENTION

The present subject matter relates generally to surgical suction devices, and more particularly to a removeable and replaceable tip for a suction device which facilitates the safe and effective removal of blood and other bodily fluids from a body cavity while allowing manipulation and/or management of various tissues therein.

### BACKGROUND OF THE INVENTION

During surgical operations, it is often desirable to remove fluid such as blood and other body fluids, and debris such as bone dust from the body cavity being operated on. This is commonly done with a tube connected to a suction device. However, since undesired trauma might result if tissues are drawn into the suction tube, and the suctioned fluid may also contain solid particles, it is customary to lay down a sponge in the body cavity and press the end of the suction tube against the sponge, so that any fluid is drawn into the suction tube through the sponge without picking up tissue, preventing clogging of the suction tube and preventing contact between delicate living tissue(s) and the end of the suction tube. Such a procedure is often time-consuming and inconvenient, and the separate sponges can easily be left in the body cavity after surgery.

Moreover, there is often a need for a surgeon to move, shift, separate and/or otherwise manipulate anatomical tissues during a surgical procedure. In many cases, manipulation of these tissues can cause and/or induce the release of blood and/or other body fluids, into the surgical cavity, often obscuring the surgeon's view of the surgical field, or damage delicate anatomic structures. It would be desirable to have a surgical instrument capable of safely manipulating anatomical tissues while concurrently removing fluids during a surgical procedure.

Accordingly, there is need for further improvement in surgical instruments and tools, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

Because the direct application of strong negative pressure can damage delicate anatomic structures such as nerves, brain tissue, dura, veins, arteries, eyes, cysts, and tumors, especially if such tissues are drawn into a suction tube, the present invention desirably isolates those tissues via a permeable sponge from the suction tube. By adding a protective sponge material at the tip of the suction tube, the tissue is protected from too much negative pressure.

Various embodiments herein include removeable and/or replaceable tips for a suction device which allow the safe and effective removal of blood and other bodily fluids from a body cavity while facilitating the manipulation and/or management of various anatomical tissues within the surgical field. Surgical suction is used to clear the operative field of fluids and debris such as blood, irrigation fluid, bone dust, lymph fluid, pus, cerebrospinal fluid, and soft tissue and others. Strong negative pressure can damage delicate anatomic structures such as nerves, brain and small blood vessels, potentially leading to severe surgical complications. Suction as high as −600 mmHg is typical in the operating room.

In one exemplary embodiment, a flexible silicon or similar material tip is provided having a lumen extending therethrough, with a distal portion of the tip securing and retaining a semi-flexible sponge or other permeable structure, wherein the sponge and/or tip is/are sufficiently rigid to allow the sponge/tip to contact and manipulate tissue(s) while concurrently providing for the removal of blood and other bodily fluids through the sponge/tip lumen. The tip can be attached to a generally rigid suction tube of a standard surgical suction device, with the suction tip being capable of rapid and convenient removal and/or replacement.

In various embodiments, a surgical sponge material may be built into the tip of the suction catheter either as a permanent feature or as a removable filter tip held by compression, bayonet type and/or other fitting. Ideally, the sponge tip is constructed of materials already approved for surgical use (Codman, American Surgical, Medline, Weck-cel) such as spun rayon, cotton, cellulose and more. The material will desirably allow clearance of fluids by suction with the tip vacuum pressure decreased at the fluid-tissue interface.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIGS. 1A and 1B depict various views of one exemplary embodiment of a suction tool tip;

FIG. 1C depicts an exploded view of the suction tool tip of FIGS. 1A and 1B;

FIG. 2A depicts a perspective view of one exemplary embodiment of a sponge tip;

FIG. 2B depicts the sponge tip of FIG. 2A in a folded configuration;

FIGS. 3A and 3B depict perspective views of one exemplary embodiment of a tip body;

FIG. 3C depicts a cross-sectional view of the tip body of FIG. 3B;

FIGS. 4A and 4B depict perspective and cross-sectional views of a views of a suction tool tip attached to a suction tube; and

FIG. 5 depicts an alternative embodiment of a tip body.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals may represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,”unless expressly specified otherwise.

In some embodiments, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may comprise various surgical materials, including silicone, rubber, latex, or any other biocompatible material, and/or any combination(s) thereof.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it should be readily apparent that more than one component, device and/or article may be used in place of a single component, device and/or article, unless otherwise noted. Similarly, where more than one component, device and/or article is described herein, it should be readily apparent that a single component, device and/or article may be used in place of the more than one component, device and/or article, unless otherwise noted. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

While performing certain surgical procedures, especially in the brain and spine, a surgeon often places a neurosurgical cottonoid sponge, also called a cottonoid or neuro pattie, or pattie, over or against the underlying tissue to protect the tissue, absorb body fluids, and irrigation fluids. The surgeon frequently uses a suction aspirator device, often called a suction cannula, or suction tip, or suction catheter, or suction tube, against the surface of the cottonoid sponge for purposes of protection of tissue, and for use as a dissecting tool. Suctioning against such barrier is a common practice in many surgical specialties, especially neurosurgery and spine surgery, but also plastic surgery, and head and neck surgery, and hand surgery. As the surgeon proceeds through the surgical field, the sponge will often need to be moved, repositioned, or replaced. The invention places the cottonoid sponge at the tip of the suction cannula allowing the surgeon to safely suction body and irrigation fluids away from the surgical field, preventing injury to delicate tissues. The sponge tip is of the same material as commercially available cottonoid sponge, or of permeable materials not yet available or considered for this purpose. It is held in place by a flexible connector to the tip of the suction tube or similar device. The connector can be designed to friction fit onto the end of the cannula, so that it can be removed when not needed during surgery as well as to allow replacement of the suction tool tip if it becomes blocked by tissue, blood, clot, bone, dust, etc. In another embodiment, the connector may also have a ridge, tab or indent to prevent loosening of the cannula tip, with the tip optionally including a mating feature to accommodate the ridge, tab or indent.

Various tip shapes can be provided with the invention to desirably prevent tissue damage while allowing for desired suction. For example, a sponge such as a cellulose eye spear or a cotton or rayon neuro-cottonoid patty may be incorporated to protect tissue. Various embodiments described herein can integrate a permeable material and/or sponges into a suction tube tip, either as a permanent or removable feature. The disclosed suction tip could comprise cotton, rayon, cellulose, or any other sponge material currently available in surgery and dentistry, or even materials in development or not yet invented. If desired, the tip material will incorporate a radio-opaque marker for identification in a surgical field.

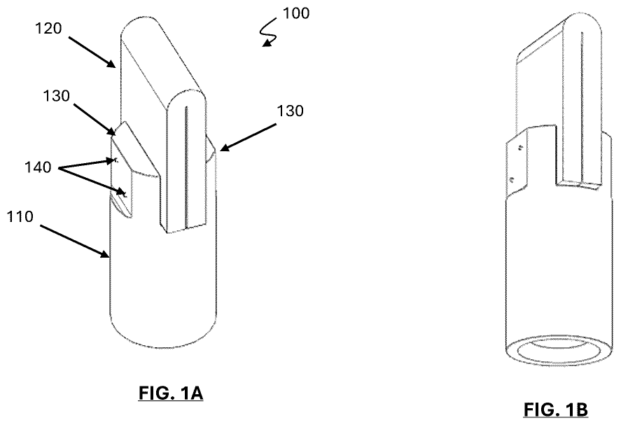

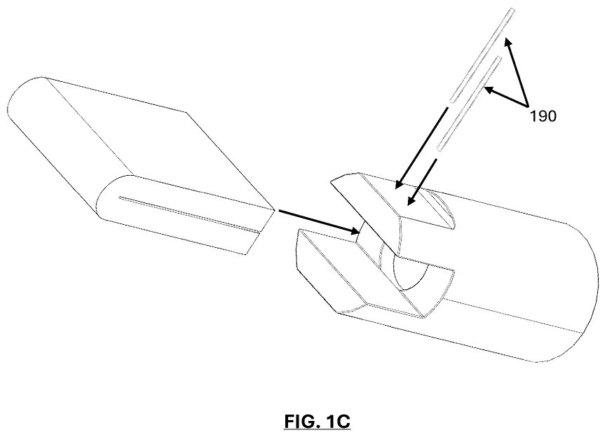

FIGS. 1A and 1B depict one exemplary embodiment of a suction tool tip 100 constructed in accordance with teachings of the present invention. The tool tip 100 includes a tip body 110 and a permeable sponge tip 120, with the sponge tip secured between projections or tabs 130. The sponge tip is optionally secured between the tabs 130 by one or more sutures or staples 140.

FIG. 1C depicts an exploded view of the suction tool tip 100 of FIGS. 1A and 1B, showing how the sponge tip 120 can be inserted between the tabs 130, and the sutures 140 extending through the tabs 130 and sponge tip 120. As noted herein, the sponge part of the suction tip can be connected to the tip body using a variety of techniques, including but not limited to suturing, stitching, stapling, gluing, or the like.

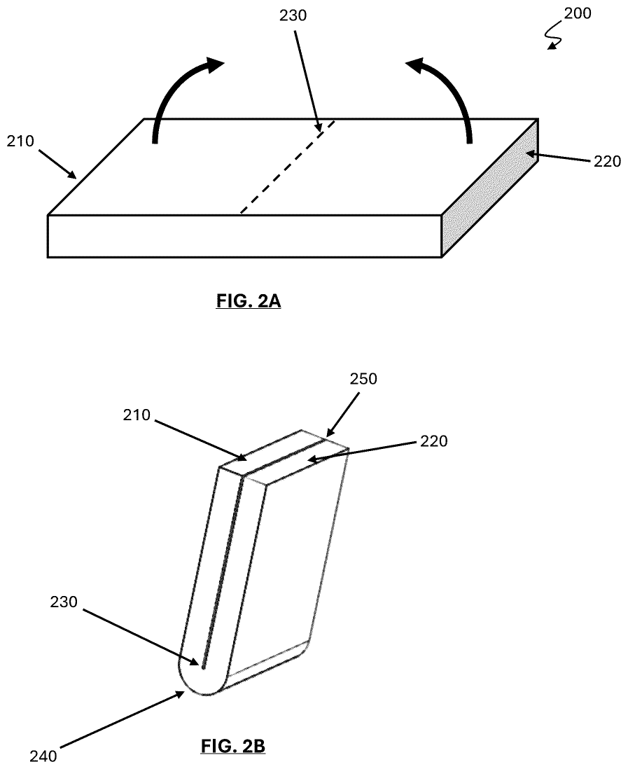

FIG. 2A depicts a perspective view of one exemplary embodiment of a sponge tip 200 in an unfolded configuration, with the arrows showing how the “leaves” or ends 210 and 220 of the sponge tip can be folded together into the configuration shown in FIG. 2B. The sponge tip 200 can comprise cotton, rayon, cellulose, and/or any other sponge material currently available in surgery and/or dentistry, as well as other materials known to those of skill in the art or developed in the future. The sponge tip 200 can be folded or otherwise manipulated along a fold line 230 to create a generally rounded, blunt tip 240. Desirably, the ends 210 and 220 can fit between the tabs of the tip body 110, with the blunt tip 240 extending distally from the tip body 110. Folding of the sponge tip 200 will desirably impart varying degrees of rigidity and/or stability to the sponge tip, and may also create a slot or recess 250 within the sponge tip that can draw fluids through the sponge body 200 and/or into the sponge tip 200 in a desired manner.

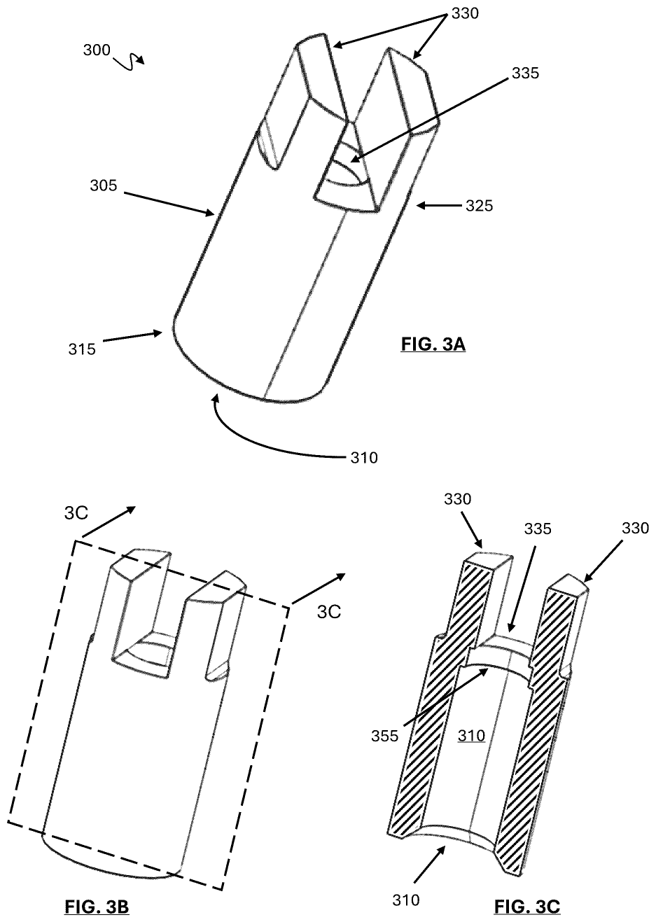

FIGS. 3A and 3B depict perspective views of one exemplary embodiment of a tip body 300. In various embodiments, the tip body 300 can comprise silicon, plastic, elastomers, and/or a variety of surgically safe materials know to those of ordinary skill in the art. The tip body 300 can comprise a generally cylindrical body 305 having a lumen 310 extending therethrough. A proximal end 315 of the tool tip 300 can include a proximal opening 320, with a distal end 325 of the tip body 300 having one or more support elements or tabs 330. A distal opening 335 of the lumen 310 is desirably positioned proximate a base of the tabs 330. As best seen in the cross-sectional view of FIG. 3C, the lumen 310 can include a lip 355 formed integrally therein. Desirably, at least a portion of the sponge tip will extend and/or be positioned across the distal opening 335, whereby a suction force passing through the lumen will pass into the sponge tip and draw fluids from the surgical field through the sponge, through the lumen and into the suction tube.

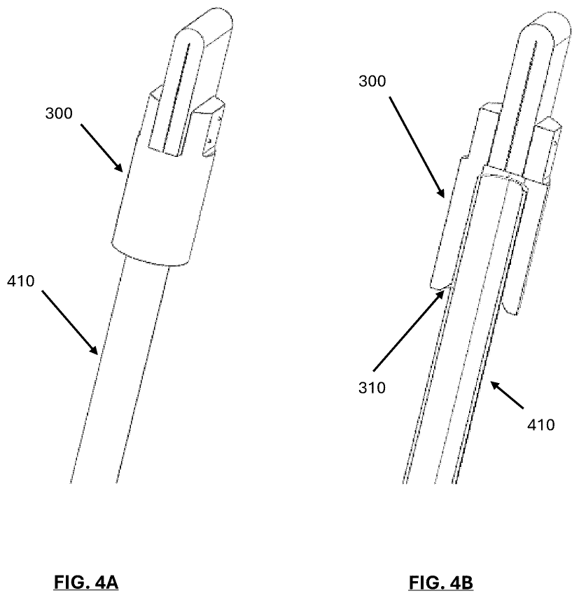

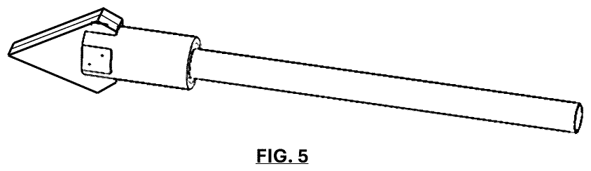

FIGS. 4A and 4B depict perspective and cross-sectional views of a views of a suction tool tip 300 attached to a suction tube 410 of a standard surgical suction device such as a Frazier or Fukushima suction tip (not shown). In use, the suction tube 410 will desirably fit into the lumen 310 of the tip body 300. In the exemplary embodiment, the lumen 310 of the tip body 300 is sized and configured slightly smaller in diameter than the outer diameter of the suction tube 410, such that flexible material of the tip body 300 must expand somewhat to accommodate the suction tube, thereby retaining the tip body 300 on the suction tube 410. If desired, a lip 355 (see FIG. 3C) can be provided within the lumen 310 which the distal end of the suction tube 410 can abut when fully seated. While the tip body is shown as engaged with the suction tube using and expandable compression and/or friction fitting arrangement, it should be understood that other engagement and retention arrangements, such as a barbed adapter, a bayonet fitting, a screw fitting and/or a luer fitting, as well as other adapter configurations known in the art, could be alternatively utilized. In other embodiments, the connector may also incorporate a ridge, tab or indent (not shown) to prevent loosening of the cannula tip, with the tip optionally including a mating feature to accommodate the ridge, tab or indent.

As best seen in FIG. 1C, one or more sutures 190, pins or other securement devices can extend through the permeable sponge tip 120 and into/through the tabs 130 of the tip body 300. Desirably, these securement devices will retain the sponge tip 120 on the device, alleviating any concern for the sponge tip to be left within the surgical field upon completion of the surgical procedure.

Surgical sponges are often embedded with some technology to find within surgical wounds or body cavities. Generally, sponges are embedded with a radio-opaque marker. If desired, the suction tool tip and/or components thereof can incorporate one or markers or other radiopaque elements which can be visualized and/or detected using non-invasive visualization methods. Such markers could be helpful in ensuring that the sponge tip is not inadvertently left within the surgical field upon completion of the surgical procedure. Desirably, tip body and sponge tip will be securely fashioned to avoid separating from the sponge tip and the suction tip and getting lost in the patient. The connector and sponge tip itself could also be made with a radiopaque marker embedded, to make it identifiable under radiographic examination.

It should be understood that the tip bodies and sponges described herein can be made and/or assembled in a variety of sizes, shapes and/or configurations to accommodate currently used suction tips and suction capacity. For example, FIG. 5 depicts one alternative permeable sponge tip configuration which can be used with the tip body.

The present invention provides various components, devices, systems and methods for treating various anatomical structures of the human and/or animal anatomy.

In accordance with another aspect of the present subject matter, various methods for manufacturing and/or assembling a suction tool tip as set for within any of the details described with the present application are provided.

If desired, a suction tool tip constructed in accordance with the various teaching herein could be constructed from a variety of modular components, including modular components comprising different materials. If desired, such modular components could be provided in a kit form for selection and/or assembly in a surgical theatre and/or in situ during a surgical procedure. If desired, various components described herein may be removable and replaceable.

While embodiments and applications of the present subject matter have been shown and described, it would be apparent that other embodiments, applications and aspects are possible and are thus contemplated and are within the scope of this application. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A surgical tool for use in a body cavity comprising:
a body having a proximal body end, a distal body end and a lumen extending through the body from the proximal body end to the distal body end, the body comprising a flexible material,
a proximal end of the lumen dimensioned to receive a suction tube tip of a surgical suction tool therein,
the distal body end having a plurality of projections extending distally therefrom, a distal end of the lumen positioned between at least two of the plurality of projections,
a surgical sponge positioned between the at least two of the plurality of projections at a location proximate to the distal ends of the lumen, the surgical sponge being attached to at least one of the plurality of projections;
wherein, when a suction force is applied by the surgical suction tube, the suction force draws a fluid from the body cavity through the surgical sponge and into the suction tube.

2. The surgical tool of claim 1, wherein the lumen includes a reduced diameter section proximate to the distal end of the lumen.

3. The surgical tool of claim 1, wherein the lumen is sized smaller in diameter than an outer diameter of the suction tube tip of a surgical suction tool, wherein the flexible material surrounding the lumen elastically expands to accommodate the suction tube tip.

4. The surgical tool of claim 3, wherein the body is retained on the suction tube tip by frictional engagement between an inner wall of the lumen and an outer wall of the suction tube tip.

5. The surgical tool of claim 1, wherein at least one of the body and the surgical sponge is impregnated with a radio-opaque marker.

6. The surgical tool of claim 1, wherein the surgical sponge includes a sponge portion that is sufficiently rigid to allow the sponge to contact and manipulate tissue.

7. The surgical tool of claim 1, wherein at least a portion of the surgical sponge extends distally from a distal end of the plurality of projections.

8. The surgical tool of claim 1, wherein at least one suture extends through the at least two of the plurality of projections and the surgical sponge.

9. The surgical tool of claim 7, wherein the surgical sponge includes a folded portion positioned distally from the distal end of the plurality of projections.

10. The surgical tool of claim 7, wherein the folded portion of the surgical sponge comprises a generally rounded, blunt tip.

11. The surgical tool of claim 7, wherein the folded portion creates a slot or recess in fluid communication with the lumen.

12. A removeable tip for a surgical suction tube, comprising:
an elongated body having a proximal end and a distal end, with a lumen extending therebetween,
at least a portion of the body comprising a flexible material,
the lumen including a proximal lumen end sized and configured to receive a distal end of the surgical suction tube,
the elongated body including at least one tab extending distally from the elongated body, a base of the at least one tab positioned proximal to a distal lumen end of the lumen, and
a surgical sponge attached to the at least one tab, wherein at least a portion of the surgical sponge extends across the distal lumen end.

13. The removeable tip of claim 12, wherein the surgical sponge includes first end, a second end and folded portion positioned therebetween, and the first and second ends are positioned proximate to the distal lumen end.

14. The removeable tip of claim 12, wherein the elongated body includes a plurality of tabs extending distally from the elongated body, and the surgical sponge is positioned between the plurality of tabs.

15. The removeable tip of claim 14, wherein at least a portion of the surgical sponge extends distally beyond at least one distal end of the plurality of tabs.

16. The surgical tool of claim 12, wherein the lumen is sized smaller in diameter than an outer diameter of the surgical suction tube, wherein the flexible material of the body is capable of expanding to accommodate a distal end of the surgical suction tube.

17. A surgical suction tube kit having a plurality of removable and replaceable tip elements, comprising:
an elongated hollow cylindrical tube connected to a vacuum source,
a plurality of removeable tips for placement on a distal end of the elongated hollow cylindrical tube, each of the plurality of removeable tips comprising an elastic body having a proximal end, a distal end and a lumen extending therethrough, the lumen sized and configured with a smaller diameter than an outer diameter of the distal end of the elongated hollow cylindrical tube, wherein the elastic body is capable of stretching the lumen to a larger diameter to accommodate the outer diameter of the distal end of the elongated hollow cylindrical tube therein,
each of the plurality of removeable tips including a surgical sponge attached proximate to the distal end thereof, wherein when one of the removeable tips is attached over the distal end of the elongated hollow cylindrical tube, a vacuum from the vacuum source passes through the surgical sponge and draws fluid from a body cavity through the surgical sponge and into the elongated hollow cylindrical tube.

18. The surgical suction tool kit of claim 17, wherein the plurality of removeable tips comprises a plurality of different sized lumens.

19. The surgical suction tool kit of claim 17, wherein the plurality of removeable tips comprises a plurality of different sized surgical sponges.

20. The surgical suction tool kit of claim 17, wherein each of the plurality of removeable tips comprises at least one tab extending from the elastic body, and the surgical sponge is attached to the at least one tab.
